(19) United States
(12) Patent Application Publication
(10) Pub. No.: US 2011/0206683 A1
(43) Pub. Date: Aug. 25, 2011

(54) PRESERVATION MIXTURE AND USE THEREOF

(75) Inventors: Cornelis Wilhelmus Van Ingen, Utrecht (NL); Chen Shu-hui Tan, Amsterdam (NL)

(73) Assignee: DE STAAT DER NEDERLANDEN, VERT. DOOR DE MINISTER VAN VWS, Den Haag (NL)

(21) Appl. No.: 13/125,592

(22) PCT Filed: Oct. 22, 2009
(86) PCT No.: PCT/NL2009/050638

§ 371 Date: Apr. 22, 2011

Related U.S. Application Data
(60) Provisional application No. 61/107,495, filed on Oct. 22, 2008.

(30) Foreign Application Priority Data
Oct. 22, 2008 (EP) 08167265.1

(51) Int. Cl. A61K 39/42; A61K 39/07; A61K 39/05; C12N 5/071; A61K 39/13; A61P 35/00; A61P 31/04; A61P 31/12

(52) U.S. Cl. 424/147.1; 424/246.1; 424/245.1; 435/364; 424/217.1

(57) ABSTRACT

The invention relates to a preservation composition, a formulation comprising a preservation mixture of glutamate, a saccharide, and a polymer. Said preservation mixture is advantageously used for the preservation of a biological compound.

### FIELD OF THE INVENTION

The invention relates to a preservation composition, a formulation comprising a preservation mixture of glutamate, a saccharide, and a polymer. Said preservation mixture is advantageous to be used for the preservation of a biological compound.

### BACKGROUND OF THE INVENTION

The long-term storage of biological compounds poses a unique challenge, considering that these compounds are usually fragile and environmentally vulnerable on our planet. Very few hydrated biological compounds are sufficiently stable to allow them to be isolated, purified and stored at room temperature as a solution for anything more than a very short period of time.

Both commercially and practically, storage of biological compounds in dry form carries with it enormous benefits. Successfully dried reagents, materials and tissues have reduced weight and require reduced space for storage notwithstanding their increased shelf life. Room temperature storage of dried materials is moreover cost effective when compared to low temperature storage options and the concomitant cost. There exist already some current technologies for producing dried biological compounds. One of the oldest and commonly used technique is freeze-drying. For a long period of time freeze-drying was seen as more of an Art than a Science, which hindered a scientific approach and research.

WO 01/37656 discloses a way of preserving a biological compound, wherein a non-reducing derivative of a monosaccharide is present. Such compounds are found attractive for preserving biological compounds such as viruses and cells. However, the use of such compounds has at least two drawbacks: such compounds should be specifically chemically synthesized and as such are expensive. In addition, such compounds have a very low glass transition temperature, which means they increase the risk of crystallization of compounds during the drying process, which may affect the stability of the biological compound.

The inventors surprisingly found that a mixture of simple materials such as glutamate, a saccharide or sugar alcohol and a polymer could be advantageously used as a preservation mixture for any biological compound.

### DESCRIPTION OF THE INVENTION

### Preservation Mixture

In a first aspect, there is provided a preservation mixture comprising glutamate, a saccharide and a polymer, wherein the saccharide is not a non-reducing derivative of a monosaccharide. In the context of the invention, a preservation mixture is called a preservation composition when a biological compound is present.

As used herein, the term “preservation” preferably means that degradation of a biological compound as identified herein by chemical pathways (such as oxidation, hydrolysis or enzymatic action) and physical pathways (such as denaturation, aggregation, gelation), for example, does not exceed an acceptable level. In other words, at least a level of biological activity or viability and/or a level of original function or structure sufficient for the intended commercial application of the biological compound is retained after drying and/or subsequent storage.

In a preferred embodiment of the invention, a preservation mixture preserves at least 0.1%, at least 0.5%, at least 1%, at least 5%, at least 10%, at least 20%, at least 30%, at least 40%, at least 50%, at least 60%, at least 70%, at least 80%, at least 85%, at least 90%, at least 95% or more of a biological activity or of viability is retained upon rehydration after freeze-drying and subsequent storage for one, or two or three or four days, one week, two weeks, three weeks, four weeks, five weeks, six weeks, seven weeks, eight weeks, nine weeks, ten weeks or more at 37° C. Depending on the identity of the biological compound, the skilled person will know which assay is to be used for assessing an activity of said biological compound. Viability is preferably assessed by determination of the colony forming units (CFU) by counting of colonies on medium formed after 5 weeks incubation at 37° C.

In another preferred embodiment of the invention, a preservation mixture preserves at least 1%, at least 5%, at least 10%, at least 20%, at least 30%, at least 40%, at least 50%, at least 60%, at least 70%, at least 80%, at least 85%, at least 90%, at least 95% or more of the structure and/or function is retained upon rehydration after storage for one, or two or three or four days, one week, two weeks, three weeks, four weeks, five weeks, six weeks, seven weeks, eight weeks, nine weeks, ten weeks or more at 37° C. Depending on the identity of the biological compound, the skilled person will know which assay is to be used for assessing a structure and/or function of said biological compound. Antigen structure is preferably assessed by ELISA or Biacor analysis. Secondary and tertiary structure is preferably assessed by UV-, fluorescence, Fourier Transformed Infra Red (FTIR) and/or Circular Dichroism (CD) sprectroscopy. Immunogenicity is preferably assessed by in vivo analysis using murine models.

Within the context of the invention, a “mixture” preferably means that each of glutamate, a saccharide and a polymer are present together.

A saccharide as present in a preservation mixture of the invention is not a non-reducing derivative of a monosaccharide. The term” non-reducing derivative of a monosaccharide” is used to refer to a general class of modified sugars. A modification may be any known chemical modifications, methylated, ethylated and chlorinated derivatives being preferred. Therefore a saccharide of the invention is not a methylated monosaccharide, or an ethylated monosaccharide or a chlorinated monosaccharide. More preferably, a methylated monosaccharide is not an α or β form of said methylated monosaccharide. Even more preferably a methylated monosaccharide is not methyl α-d-glucopyranoside. The use of such specific non-reducing derivative of a monosaccharide is disclaimed since they have some drawbacks: although such compounds were found attractive for preserving biological compounds such as viruses and cells, they have at least two drawbacks. They should be specifically chemically synthesized and as such are expensive. In addition, such compounds have a very low glass transition temperature, which means they may increase the tendency to crystallize during the drying process, which affects the stability of the biological compound to be preserved. In a particularly preferred embodiment of the invention, a preservation mixture is provided as defined herein before, wherein the mixture does not comprise methyl α-d-glucopyranoside. More preferably the mixture does not comprise a methylated monosaccharide, or an ethylated monosaccharide or a chlorinated monosaccharide. Still more preferably, the mixture does not comprise a non-reducing derivative of a monosaccharide.

Without wishing to be bound by any theory, the inventors believe that in order to optimize the preservation of a biological compound, it is crucial that the preservation mixture used has a high glass transition temperature in order to avoid crystallisation of components of the preservation mixture during freeze drying and subsequent storage . Within the context of the invention, a “high” glass transition temperature (Tg), preferably means a glass transition temperature after drying which is higher than the storage temperature. More preferably, a high glass transition temperature is higher than 0° C., or higher than 10° C., or higher than 15° C., or higher than 20° C., or higher than 25° C., or higher than 27° C., or higher than 28° C. or higher than 29° C. or higher than 30° C. or higher than 31° C. or higher than 32° C. or higher than 33° C. or higher than 34° C. or higher than 35° C. or higher than 36° C., or higher than 37° C., or higher than 38° C. or higher than 39° C. or higher than 40° C. or higher than 45° C. or higher than 50° C. or higher than 55° C. or higher than 60° C. or higher than 65° C. or higher than 70° C. or higher than 75° C. A glass is an amorphous solid state which may be obtained by substantial undercooling of a material that was initially in the liquid state. In the present invention, a glass is obtained during the freeze-drying process of a biological compound using a preservation mixture of the invention. Diffusion in vitrified materials, or glasses, occurs at extremely low rates (e.g. microns/year). Consequently, chemical or biological changes requiring the interaction of more than one moiety are practically completely inhibited. Glasses normally appear as homogeneous, transparent, brittle solids, which can be ground or milled into a powder. Above a temperature known as the glass transition temperature (Tg), the viscosity drops rapidly and the glass becomes deformable and the material turns into a fluid at even higher temperatures. We believe that the optimal benefits of vitrification for long-term storage may be secured only under conditions where Tg is greater than the storage temperature. The Tg is directly dependent on the amount of water present in the glass, and may therefore be modified by controlling the level of hydration; the less water, the higher the Tg. The inventors found that a preservation mixture comprising glutamate, a saccharide and a polymer has an attractive high Tg as defined earlier herein. Furthermore, the inventors clearly and unambiguously demonstrated (see the experimental part) that the use of such preservation mixture having such a high Tg leads to the obtaination of a biological compound which is efficiently preserved. The meaning of the word “preserved” is the same as for the word “preservation” as earlier defined herein. In addition, drying processes, like a freeze-drying process, can be designed to be more efficient (i.e. shorter time, temperature of the process may be higher) with such a formulation. A freeze-drying process of the invention is later defined herein.

Each constituent of a preservation mixture of the invention is now extensively identified below.

### Glutamate

Glutamate is preferably sodium glutamate, potassium glutamate, ammonium glutamate, calcium diglutamate, magnesium diglutamate, glutamic acid. More preferably sodium glutamate is used. In a preferred embodiment, glutamate is present in a preservation composition in an amount which is ranged between about 0.05% w/v and about 60% w/v, more preferably between about 0.2 and about 55% w/v, more preferably between about 0.5 and about 50% w/v, even more preferably between about 1 and about 45% w/v, even more preferably between about 1.5 and about 40% w/v, even more preferably between about 2 and about 40% w/v, even more preferably between about 2 and about 35% w/v, even more preferably between about 2 and about 30% w/v, even more preferably between about 2 and about 25% w/v, even more preferably between about 2 and about 20% w/v, even more preferably between about 4 and about 15% w/v, even more preferably between about 4 and about 10% w/v. Very good results were obtained using about 5% w/v (see the example). Therefore in a preferred embodiment, about 5 to about 10% w/v of sodium glutamate is present in a preservation composition. More preferably, 5 to 10% w/v/ of sodium glutamate is present in a preservation composition. Without wishing to be bound by any theory, the inventors believe that glutamate may be able to bind water within a composition comprising a biological compound and a preservation mixture of the invention during the drying process. As a result, water may be removed in a later stage and/or in a relatively slow manner during freeze-drying, i.e. by sublimation under vacuum. The fact that water may be retained for a longer time and/or that it may be removed in a relatively low manner may explain why such a preservation mixture is highly effective.

### Mono-, Di- or Oligo Saccharide

A saccharide is further present in a preservation mixture of the invention, said saccharide not being a non-reducing derivative of a monosaccharide as mentioned in WO 01/37656. A saccharide as present in a preservation mixture may be a mono-, di- or oligosaccharide. Examples of suitable monosaccharides include glucose, mannose, fructose, xylose, galactose, ribulose, arabinose, etc. Examples of suitable disaccharides include trehalose, sucrose, lactose, maltose, etc. Examples of suitable oligosaccharides include fructooligosaccharide, galacto-oligosaccharide, mannan-oligosaccharide, etc . . . Preferably a saccharide is a mono- or di-saccharide. More preferably, a disaccharide is used. Even more preferably, trehalose is used as a disaccharide. Monosaccharides and disaccharides are believed to have a better stabilizing effect on biological material due to their small molecular size, which may result in a better interaction with a given biological material. Trehalose is highly preferred as a disaccharide since it has a relatively high Tg (pure trehalose has a Tg of about 121° C.).

In a preferred embodiment, a saccharide is present in a preservation composition in an amount which is ranged between about 0.05 and about 60% w/v, more preferably between about 3 and about 55% w/v, more preferably between about 5 and about 50% w/v, even more preferably between about 5 and about 45% w/v, even more preferably between about 5 and about 40% w/v, even more preferably between about 5 and about 35% w/v, even more preferably between about 5 and about 30% w/v, even more preferably between about 5 and about 25% w/v, even more preferably between about 5 and about 20% w/v, even more preferably between about 6 and about 20% w/v, even more preferably between about 7 and about 15% w/v. Very good results were obtained using about 7 or about 20% w/v (see the example). Accordingly, in a preferred embodiment, a preservation composition comprises about 7 or about 20% w/v trehalose. More preferably, a preservation composition comprises 7 or 20% w/v trehalose.

Without wishing to be bound by any theory, the inventors believe that a saccharide may strengthen the effect of glutamate for binding water within a matrix comprising a biological compound and a preservation mixture of the invention during the freeze-drying process as earlier explained.

### Polymer

A polymer is further present in a preservation mixture of the invention. Examples of polymers include polysaccharides, PVP, PEG. Preferably, a polymer is a polysaccharide. A polysaccharide may be dextran, HES (Hydroxy Ethyl Starch), inulin, MCC (micro crystalline cellulose), CMC (carboxy methyl cellulose), dextrin, cyclodextrin, etc. A preferred polysaccharide is HES. A polymer as defined herein is advantageous to be used in a preservation mixture since it has a relatively high Tg. In a preferred embodiment, a polymer is present in a preservation composition in an amount which is ranged between about 0.005% w/v and about 50% w/v, more preferably between about 0.2 and about 45% w/v, more preferably between about 0.5 and about 40% w/v, even more preferably between about 1 and about 35% w/v, even more preferably between about 5 and about 30% w/v, even more preferably between about 5 and about 25% w/v, even more preferably between about 5 and about 20% w/v, even more preferably between about 5 and about 15% w/v, even more preferably between about 5 and about 10% w/v, even more preferably about 10% w/v. Very good results were obtained using about 10% w/v (see the example). Therefore, accordingly, in a preferred embodiment, a preservation composition comprises about 10% w/v HES. More preferably, a preservation composition comprises 10% w/v HES. Without wishing to be bound by any theory, the inventors believe that a polymer may strengthen the effect of glutamate for binding water within a matrix comprising a biological compound and a preservation mixture of the invention during the freeze-drying process as earlier explained.

Below, we give preferred preservation compositions defining combination of three constituents together.

A preferred preservation mixture or composition is a preservation mixture or composition, wherein glutamate is sodium glutamate, and/or the saccharide is a mono-, di-, oligo- and/or a polysaccharide and/or the polymer is PVP, PEG and/or a polysaccharide. A more preferred preservation mixture or composition is a preservation mixture or composition, wherein glutamate is sodium glutamate, the saccharide is a disaccharide and/or the polymer is a polysaccharide. An even more preferred preservation mixture or composition is a preservation mixture or composition, wherein glutamate is sodium glutamate, the disaccharide is trehalose and/or sucrose, and/or the polysaccharide is dextran, HES, MCC and/or dextrin. A most preferred preservation mixture or composition is a preservation mixture or composition, wherein glutamate is sodium glutamate, the disaccharide trehalose and/or the polysaccharide HES. A preferred preservation composition is a preservation composition, wherein glutamate is present in an amount ranged between about 0 and about 60% w/v, a saccharide about 1 and about 60% w/v and a polymer about 0 and about 50% w/v. A more preferred preservation composition is a preservation composition, wherein glutamate is present in an amount ranged between about 5 and about 10%, a saccharide between about 10 and about 20% and a polymer about 10%.

### Preservation Composition

In a further aspect, there is provided a preservation composition comprising a preservation mixture as defined herein and a biological component.

The term “biological component” encompasses or is or comprises or consists of a peptide, a polypeptide, a protein, an enzyme and coenzyme, a serum, a cell, a liposome, an adjuvant, a vitamin, an antibody, and an antibody fragment. Both naturally-derived or purified and recombinantly produced moieties are included in these terms. This term also includes a lipoprotein and a post-translationally modified form, e. g., a glycosylated protein. An analogue, derivative, agonist, antagonist and a pharmaceutically acceptable salt of any of these are included in these terms. The term also includes a modified, derivatives or non-naturally occurring peptide having D-or L-configuration amino acids.

The term “biological component” further includes or comprises or consists of any antigenic substance, capable of inducing an immune response. More particularly, an antigen may be a protein or fragment thereof expressed on the extracellular domain of a tumor (e. g., for the treatment of cancer), an allergen, or an infectious agent (e. g., virus or bacteria) or portion thereof (e.g. subunit, whole inactivated virus, inactivated bacteria, VLP, toxin). Therefore, the term encompasses an epitope of a pathogen, said epitope being recognized by the immune system to induce an immune response. The term also encompasses a vaccine. A vaccine refers to a peptide comprising an epitope as earlier defined herein, being used for a particular type of immunization, wherein the peptide originates or derives from an infectious agent (or any part thereof), which is administered to a mammal to establish resistance to the infectious disease caused by the agent. Vaccines may include viruses, bacteria and parasites, viral particles and/or any portion of a virus or a micro-organism including an infectious disease agent or pathogen, including proteins and/or nucleic acids, which may be immunogenic and therefore useful in the formulation of a vaccine. Preferred bacteria include Helicobacter, such as H. pylori, Neisseria, such as N. meningitidis, Haemophilus, such as H. influenza, Bordetella, such as B. pertussis, Chlamydia, Streptococcus, such as Streptococcus sp. serotype A, Vibrio, such as V. cholera, Gram-negative enteric pathogens including e.g. Salmonella, Shigella, Campylobacter and Escherichia, as well as antigen from bacteria causing anthrax, leprosy, tuberculosis, diphtheria, Lyme disease, syphilis, typhoid fever, and gonorrhea. Preferred bacteria belongs to a Bordetella or a Neisseria species. More preferred Bordetella species include Bordetella pertussis, Bordetella parapertussis, or Bordetella bronchiseptica. More preferred Neisseria species includes Neisseria meningitidis. A parasite may be a protozoan, such as Babeosis bovis, Plasmodium, Leishmania spp. Toxoplasma gondii, and Trypanosoma, such as T. cruzi. Other pathogens may be eukaryote. Preferred eukaryotes include a fungus. More preferred fungi are yeast or filamentous fungus. An example of a preferred yeast belongs to a Candida species. Preferred fungi include Aspergillus sp., Candida albicans, Cryptococcus, such as e.g C. neoformans, and Histoplasma capsulatum. Preferred viruses include but are not limited to any virus, which is able to induce a condition or a disease in a mammal. Preferably the mammal is a human being. Viruses of human beings include: Retroviridae such as Human Immunodeficiency virus (HIV); a rubellavirus; paramyxoviridae such as parainfluenza viruses, measles, mumps, respiratory syncytial virus, human metapneumovirus; flaviviridae such as yellow fever virus, dengue virus, Hepatitis C Virus (HCV), Japanese Encephalitis Virus (JEV), tick-borne encephalitis, St. Louis encephalitis or West Nile virus; Herpesviridae such as Herpes Simplex virus, cytomegalovirus, Epstein-Barr virus; Bunyaviridae; Arenaviridae; Hantaviridae such as Hantaan; Coronaviridae; Papovaviridae such as human Papillomavirus; Rhabdoviridae such as rabies virus. Coronaviridae such as human coronavirus; Alphaviridae, Arteriviridae, filoviridae such as Ebolavirus, Arenaviridae, poxviridae such as smallpox virus, and African swine fever virus. A Measles virus and an influenza virus are preferred viruses.

The term “biological component” also encompasses or is or comprises or consists of viral, bacterial and yeast-derived vectors useful in transformation of cells. Such vectors may be used for gene therapy as well as molecular biology and genetic engineering. The term “biological component” also encompasses or is or comprises or consists of a virus like particle, a virosome, a liposome, a lipoplex.

In a most preferred aspect, the term “biological component” also includes or is or comprises or consists of a virus, prokaryotic and eukaryotic cell.

In a preferred embodiment, a preservation composition comprises as biological component a micro-organism, a virus or a protein.

In a preservation composition, a biological component is present preferably in an amount ranged between 1×10⁰ and 1×10²⁵ live and/or dead particles per ml (e.g. colony forming units, plaque forming units, tissue culture 50% infectious dose, haemagglutination units) and/or preferably in a weight ranged between 1 pg/ml and 10 g/ml.

### Method

In a further aspect, there is provided a method of preserving a biological component, wherein the method comprises the following steps:
a) adding each of the components of the preservation mixture as defined earlier herein to or mixing each of these components with a biological component to obtain a preservation composition,
b) subsequently, drying the obtained preservation composition (e.g. spray drying, air drying, freeze drying, spray-freeze drying).

In a preferred method, the step of drying is conducted by air drying, desiccation, vacuum drying, freeze drying or a combination thereof. More preferably drying is performed by freeze drying.

### Step a

Each constituent of a preservation mixture as earlier identified may be mixed together, including a biological component to obtain a preservation composition. Any component of a preservation mixture including a biological component as defined herein may be sequentially or simultaneously added or mixed together. Any order of adding each of the components of the mixture and a biological component is encompassed in step a of the method. Each component of the preservation mixture may be added sequentially to a biological component. It is also encompassed to add each of the component of a preservation mixture to a solution or suspension wherein a biological component is present.

### Step b

Subsequently, an obtained preservation composition is dried. Any known drying method may be used. A drying method may be spray drying, vacuum/freeze drying or freeze drying. In a preferred embodiment, a drying method is freeze-drying. Freeze-drying processes are known to the skilled person. The shelf temperature may be of at least −50° C., or at least −40° C. or at least −30° C. or at least −20° C. However, it is preferably approximately −40 or even approximately −50° C. A preservation composition may be brought to a pressure of 100 microbar or lower. When the set pressure has been reached, the shelf temperature may be shifted to approximately −40° C. or −30° C. or −25° C. or −20° C. The primary drying step is preferably ended when no pressure rise is measured in the chamber. At that moment, the shelves are heated up to preferably 20° C. or 30° C. or 40° C. The temperature is preferably kept to this value till no pressure rise could be detected. A preferred freeze-drying process is described in the example. Due to the high Tg of some constituents of a preservation composition, the inventors believe that the efficacy of the drying process can be improved: the process is shorter. For example in one of the described examples the process could be shortened from 10 days to 9 days or even to 5 days. In addition, a biological component freeze-dried using this process sees at least one of its activities or its viability preserved as defined herein.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a preservation mixture as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “approximately” or “about” when used in association with a numerical value (approximately 10, about 10) preferably means that the value may be the given value of 10 more or less 5% of the value.

All patent and literature references cited in the present specification are hereby incorporated by reference in their entirety.

The following examples are offered for illustrative purposes only, and are not intended to limit the scope of the present invention in any way.

### DESCRIPTION OF THE FIGURES

FIG. 1 shows the glass transition temperature (Measured with a Q100 differential calorimeter, TA instruments) and the residual moisture content (RMC; measured with a Karl Fisher coulommetric titrator, Mitshubushi) of different formulations as function of the process stage (shelf temperature of the freeze dryer; ˜the energy consumption needed to lower the residual moisture content of the already stabilised cake). For long term stability in general an RMC lower than 3% is preferred.

FIG. 2 Analysis of the temperature of complete solidifying of the HES/trehalose/glutamate/BCG formulation with the freezing analyser. First the liquid preservation composition is frozen in the freeze analyser. Secondly, the temperature in the freezing analyser increases and the electrical resistance is measured. The moment, the temperature, at which the resistance changes is the temperature of complete solidifying. In this case the temperature of complete solidifying is −25° C.

FIG. 3 The glass transition temperatures (Tg) of different formulations after freeze drying. A formulation with HGT medium that contains 11% glucose, 2.5% polygeline and 0.005% Tween 80 in water. A formulation containing 5% sodiumglutamate. A formulation of the invention, a formulation containing 5% sodiumglutamate, 10% HES and 20% trehalose. The glass transition temperature is determined by differential scanning calorimetry using a Q100 differential colorimeter (TA instruments). Aluminum (DSC) pans were filled with 1-20 mg powder (freeze dried formulation) and DSC was performed at a scan speed of 10° C./min. Tg was determined using TA analysis software.

FIG. 4 Effect of volume decrease and BCG concentration on BCG survival.
A)—A volume of 10 ml containing 1×10⁹ cfu BCG, 5% sodiumglutamate, 10% HES and 20% trehalose was freeze dried and survival of BCG was determined.
B)—A volume of 5 ml containing 2×10⁹ cfu BCG (double concentration BCG compared to A), 5% sodiumglutamate, 10% HES and 20% trehalose was freeze dried and survival of BCG was determined.
C)—A volume of 5 ml containing 1×10⁹ cfu BCG (double concentration BCG compared to A), 5% sodiumglutamate, 10% HES and 20% trehalose was freeze dried and survival of BCG was determined.

FIG. 5 Storage stability of freeze dried BCG formulations.

After freeze drying dried BCG formulations were stored at 4, 20 and 37° C. for 4 weeks.

CFU's of reconstituted BCG formulations were determined before and after storage. Stability of the BCG formulations is expressed as the relative viability, the percentage of original CFU of the dried BCG formulation.

FIG. 6 Stabilization of Cl.tetani-seedlots.

A suspension of Cl. Tetani was pelleted and subsequently resuspended in the desired medium. As medium skimmilk from Super de boer (liquid skimmilk), skimmilk from BD Diagnostics (dissolved skimmilk powder, 7%), and 10% HES+20% trehalose+5% Na-glutamate were used. The final formulation in the ampoule consisted of 1-4×10⁵ cfu Cl. Tetani in 0.3 ml medium. In vials before and after freeze drying the colony forming units were determined. The recovery of the cfu was subsequently calculated.

FIG. 7 Stabilization of C. diphteriae-seedlots. A suspension of C. diphteriae was pelleted and subsequently resuspended in the desired medium. As medium skimmilk from Super de Boer (liquid skimmilk), skimmilk from BD Diagnostics (dissolved skimmilk powder, 7%), and 10% HES+20% trehalose+5% Na-glutamate were used. The final formulation in the ampoule consisted of 1×10⁸-2×10⁹ cfu C. diphteriae in 0.3 ml medium. In vials before and after freeze drying the colony forming units were determined. The recovery of the cfu was subsequently calculated.

FIG. 8. Growth curfe of freeze-dried C. diphteriae-seedlots. Freeze dried C. diphteriae (see also FIG. 7) was reconstituted, incubated on Stainer-medium and the optical density (OD) at 590 nm was measured during the first 30 hours.

FIG. 9. Viability of lyophilized Vero cells suspended in Smiff medium, stabilized by several formulations (10% HES+5% Na-glutamate+20% trehalose; 20% trehalose; 20% sucrose; 5% Na-glutamate; 10% Na-glutamate; H₂O) after 4 weeks of storage at 4° C.

FIG. 10. Potency of polio vaccine batches A, B, C and D as determined by a D-antigen ELISA using reference Moabs (frozen, −30° C.) and lyophilized moabs (10% HES+5% Na-glutamate+20% trehalose, 20° C.).

FIG. 11. Recovery of D-antigen content (Type 1; Type 2; Type 3) of lyophilized polio vaccine, stabilized using different formulations (10% HES+5% Na-glutamate+20% trehalose; 20% trehalose; 20% sucrose; 5% Na-glutamate; 10% Na-glutamate; H₂O).

FIG. 12. Recovery of D-antigen content (Type 1; Type 2; Type 3) of lyophilized polio vaccine, stabilized using different formulations (20% trehalose; 20% sucrose; 10% HES+5% Na-glutamate+20% trehalose; 10% HES+5% Na-glutamate+20% sucrose).

### EXAMPLES

### Example 1

### BCG for Bladder Cancer Immuno-Therapy by Instillation

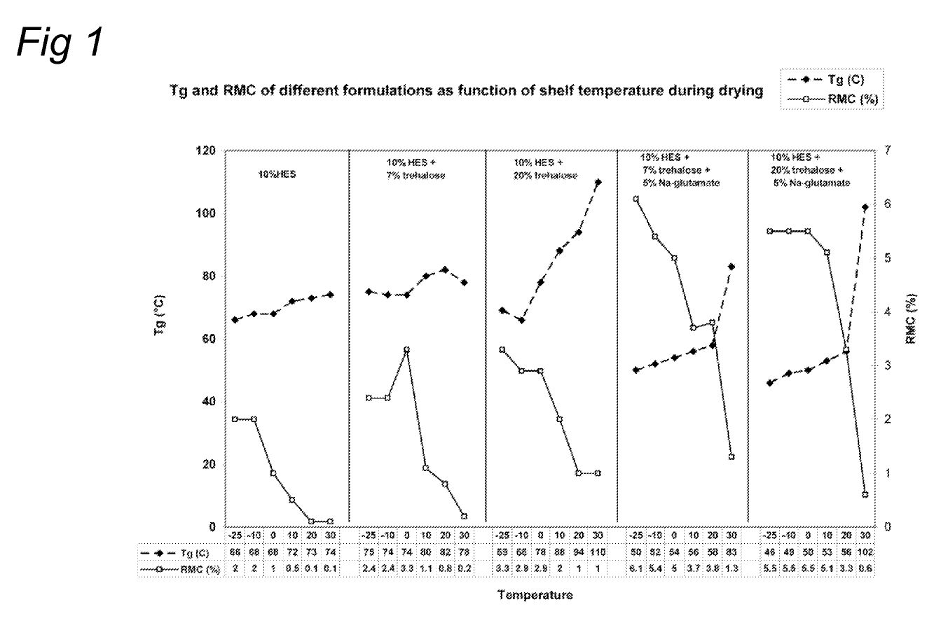

To ensure a successful immunotherapy, a minimum of 2×10⁸ live BCG bacteria is needed. With the formulation of the invention a survival rate of 80-100% is achieved after freeze drying. In addition, the dried product, cake, is very stable (physical stability) at ambient (room) temperature and higher temperatures. (see FIG. 1). Moreover, no animal components are involved in the formulation of the invention.

### 1. General Principles of the Formulation as Designed for BCG.

Freeze-drying stabilisers commonly exist in a combination of a:
Bulking agent (Mannitol, Serum Albumine, PVP, CMC, etc.). [which can have more functions]
Glassformer which is mostly a saccharide
For viruses and proteins a buffer is necessary to assure the correct pH before, during and after freeze-drying (because of the pH shift during freezing).

Typical proportions are 5 to 7% glassformer and 7 to 10% bulking agent. With a preservation mixture comprising 10% HES+20% Trehalose+5% sodium glutamate we obtained the best results. This is probably due to the fact that the 5% concentration of Sodium Glutamate binds the water severely so that it comes off from the freezing stabilised matrix in a later stadium of the freeze-drying process (see FIG. 1).

### 2. Method

### Formulation of the Preservation Mixture

Weight: 10 g HES
5 to 20 g Trehalose
5 g sodium glutamate

Each of these components was mixed together. The obtained mixture was supplemented with water to 100 ml. The obtained mixture was steam sterilised. The sterilised mixture looked like an opaque solution. The desired amount of microorganisms (in this case approx. 1×10⁹ live BCG bacteria) was added to this sterilised mixture and filled into a desired container, such as an ampoule, vial or bulk for freeze-drying. This mixture is referred to as a preservation composition according to the invention later in the example.

### Freeze-Drying Recipe

The shelf temperature of a KLEE freeze dryer was set at −30° C. or lower. However, better results were obtained at −40/−50° C. The pressure of the freeze dryer was set at 100 microbar or lower. When the set pressure had been reached, the shelf temperature was shifted to −25° C. The primary drying step was ended when no pressure rise was measured in the chamber. At that moment, the shelves were heated up to 30° C. The temperature was kept to this value till no pressure rise could be detected. Subsequently, containers were closed under vacuum or inert gas.

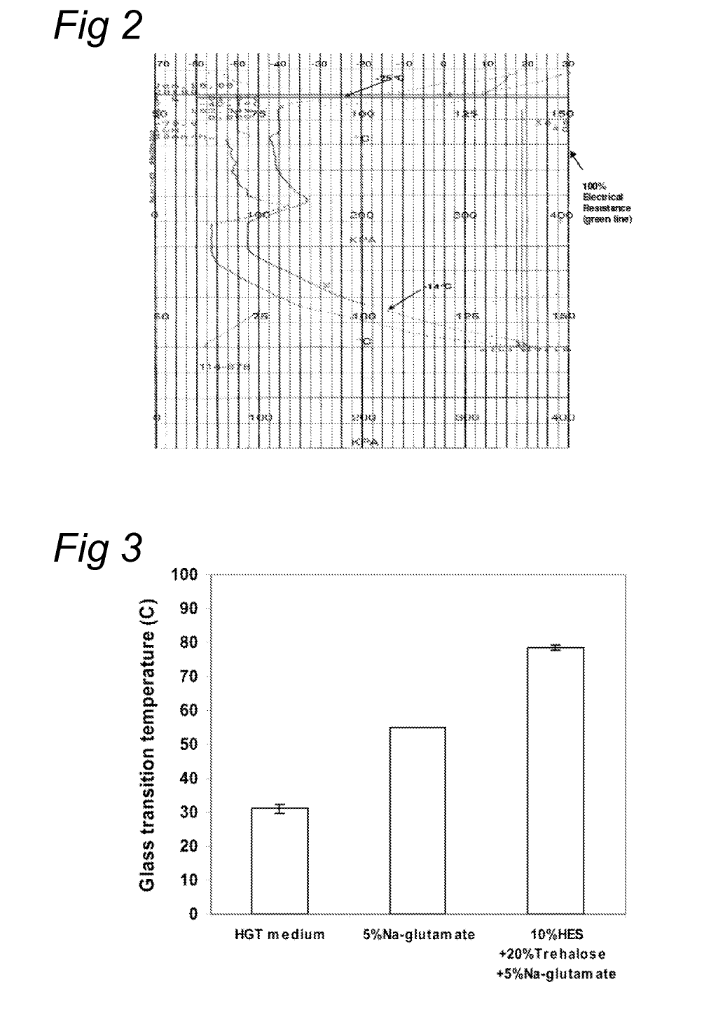

Freeze-drying is an energetically and expensive process; in order to lyophilize efficiently freeze drying in general has to be performed at the highest possible product temperature. The analysis with the freezing analyser showed that the safe temperature of the HES/trehalose/glutamate/BCG formulation (Temperature of complete solidifying) is −25° C. which is very reasonable (relative high), suitable for efficient freeze-drying (see FIG. 2).

### 3. Increased Potency of BCG

### Shortening of the Freeze Drying Process (FIG. 4)

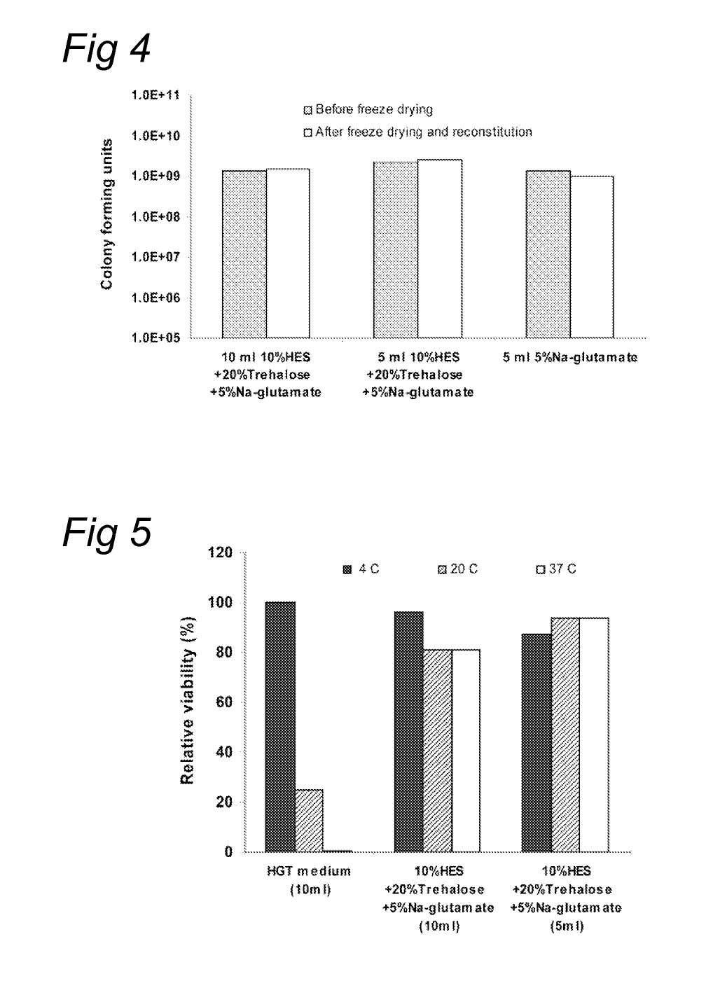

In our standard production medium of BCG, we use a 10 ml filling in order to reach the necessary potency/vial after freeze-drying. With our standard formulation we currently have a huge loss in potency after the freeze-drying process (see FIG. 5). The 10 ml filling leads to a 10 day's freeze-drying process. With present invention it is possible to shorten the freeze-drying cycle acquiring the same results by using a 5 ml filling/vial and a double concentration (cfu/ml) of live BCG. FIG. 4 shows the results of a 10 ml filling with the standard concentration with a preservation composition according to the invention, a 5 ml filling with a double concentration and a pure sodium glutamate 5 ml filling.

It is clear that a preservation composition according to the invention gives a 100% protection in both the standard and double concentration using a 5 ml filling/vial. However, in contrast to the freeze-drying cycle of the vials with 10 ml filling that took 9 day's, the freeze drying cycle of the vials with 5 ml filling took only 4 days. Although the sodium glutamate formulation with the standard concentration at 5 ml filling gave a reasonable BCG viability, no “cake” was left after freeze-drying.

### Increased Physical Stability of the BCG Product (FIG. 3)

We compared the glass transition temperature (i.e. temperature at which the cake is physically stable) after freeze-drying between the standard formulation of BCG vaccine (HGT medium), the 5% Sodium Glutamate formulation and a preservation composition according to the invention. Clearly, the standard formulation has a poor physical stability at room temperature as we know in practice. Sodium Glutamate seems good enough but the disadvantage is that there is no “cake” left after freeze-drying, making resuspention into uniform suspension impossible. A preservation composition according to the invention is physically stable up to a temperature of +75° C., far above regular storage conditions, which is in generally preferred.

### Long Term Stability (FIG. 5)

In a next experiment, we compared the log term stability of a preservation composition according to the invention and the standard HGT medium. It is common practice to evaluate the stability of freeze-dried products at elevated temperatures (so called accelerated stability test).

In this experiment, the standard formulation is compared to a preservation composition according to the invention with a 10 ml filling and a 5 ml filling with a double concentration of live BCG by measurement of the cfu directly after freeze-drying, after 4 weeks storage at 4° C., 20° C. and 37° C. The difference in stability at elevated temperatures is clear. The loss in potency with the standard HGT medium after freeze-drying is dramatic and potency loss is even more when the product is stored for 4 weeks at +20° C. (room temperature). However, no significant loss in potency is observed with a preservation composition according to the invention in standard concentration in 10 ml as well in the double concentration 5 ml fillings. Even 4 weeks at 37° C. did not seem to affect the potency of the vaccine.

### Conclusions for the BCG

1. For high (80%) survival of BCG, a mixture of 10% HES, 20% trehalose and 5% Na-glutamate gave reproducible results as visualized by stable cake structures/formation.
2 These experimental conditions resulted in <3% residual moisture content, which meets the WHO (World Health Organization) requirement.
3 With minimally 5% trehalose in the mixture, a robuust low residual moisture content (˜1%) was reached.
4 To reach a glass transition temperature of at least 55° C. (relevant for tropical areas), an end temperature after the secondary drying phase of >30° C. is required.
5 These conditions meet the stability requirement of WHO.

### Example 2

### Seedlots Diphtheriae/Tetanus

Originally seedlots of Diftheriae and Tetanus are prepared by freeze drying them with

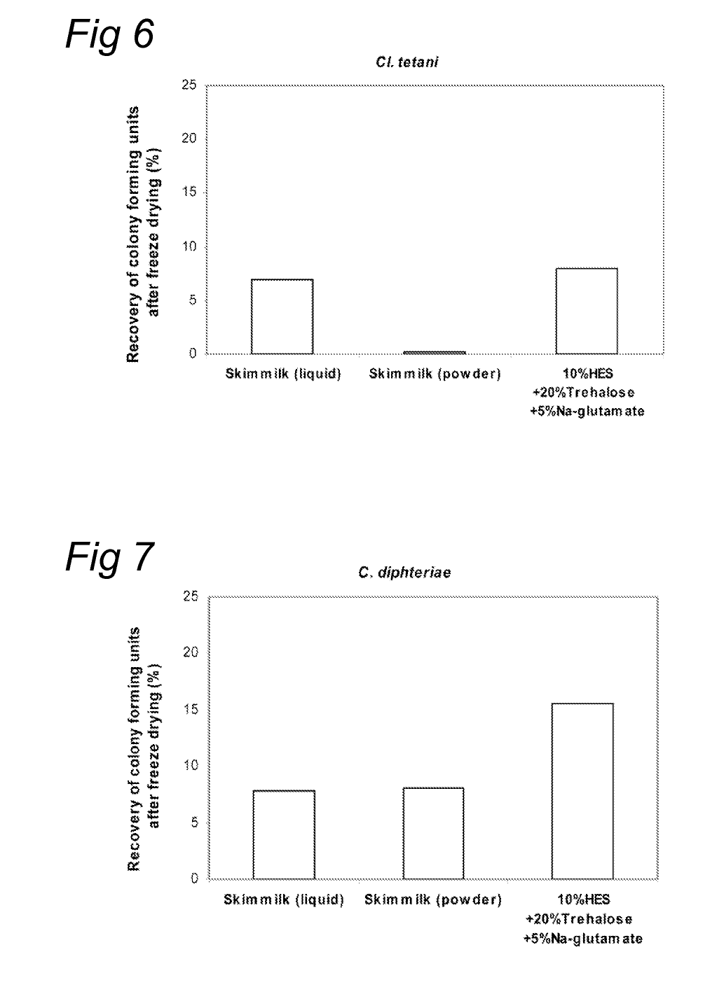

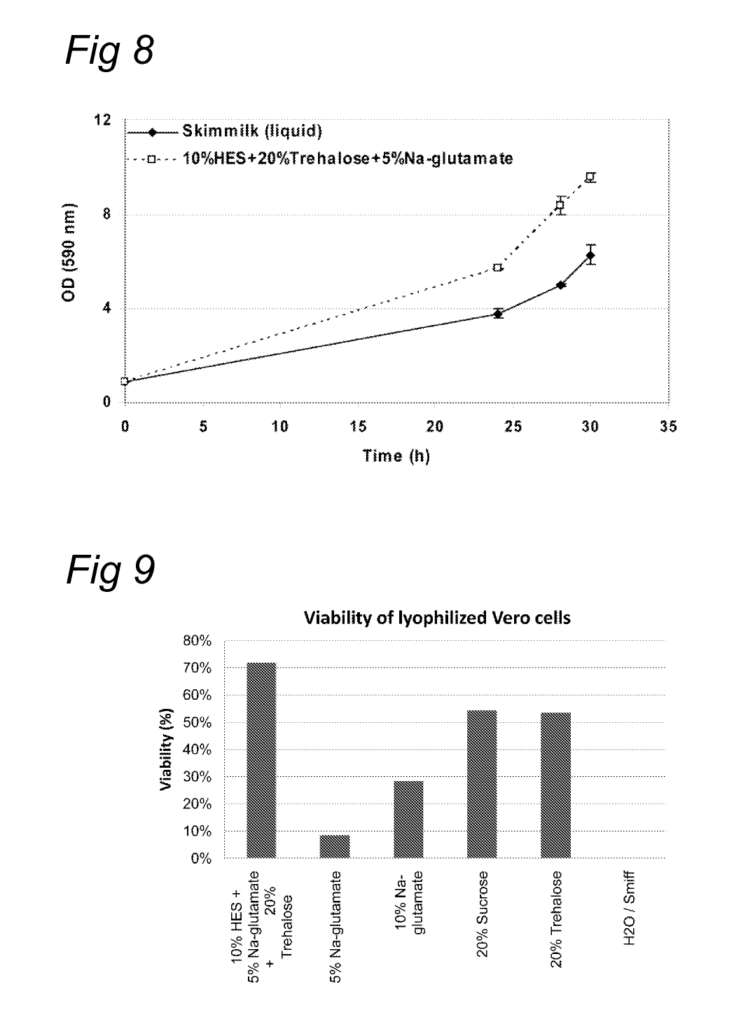

Skimmilk. However, skimmilk is from animal origin which is undesired from current regulatory point of few. As a result there is a search for “animal free” alternatives for skimmilk. A mixture of glutamate, trehalose and hydroxyethylstarch is free from animal derived components. In our lab it was found that seedlots of Cl.tetani (y-IV9 derived from Harvard strain 49205) and C. diphtheriae (CN2000 derived from Park Williams strain) could be successful freeze dried with this mixture retaining comparable or even more viability than seedlots freeze dried with skimmilk (liquid skimmilk from Super de Boer; and skimmilk prepared from skimmilk powder from BD Diagnostics) (see for results and further details FIGS. 6 and 7). In addition, C. diphtheriae stabilized by glutamate/trehalose/HES mixture showed a shorter lag-phase than C. diphtheriae stabilized by skimmilk (FIG. 8).

### Example 3

### Lyophilization of Vero Cells

In general Vero cell banks are stored frozen. The stabilization of Vero cells in the dried state using the preservation mixture of the present invention was studied. A comparison was made with other formulations. Vero cells suspended in Smiff medium (1*10̂6 cells/ml) were1:1 diluted with stabilizer solutions resulting in solutions containing 0.5*10̂6 cells/ml and:
10% HES+5% Na-glutamate+20% trehalose
20% trehalose
20% sucrose
5% Na-glutamate
10% Na-glutamate
Smiff-medium/H₂O (1:1)

Subsequently, 3 ml vials were filled with 500 μl of the resulting suspensions (A t/m F, containing 0.5*10̂6 cells/ml), placed on a pre-cooled shelf (−50° C.) of the freeze dryer for 4 hrs and lyophilized subsequently.

After lyophilization the dried Verocells were stored at 4° C. for 4 weeks and subsequently analysed on viability. Viability was assessed by counting of the percentage living cells after reconstitution of the dried Verocells with PBS 1× (Gibco #20012 pH 7.4).

As shown in FIG. 9, the viability of Vero cells lyophilized into diluted Smiff medium resulted Cinto complete loss of living Verocells. Lyophilization of Verocells using 5 or 10 Na-glutamate resulted only in 8 or 27% living Vero cells.20% Sucrose and 20% trehalose could retain up to 55% viability. The preservation mixture used in this study (10% HES+5% Na-glutamate+20% trehalose) showed the best survival rate for Verocells after lyophilization and 4 week storage at 4° C.

### Example 4

### Lyophilization of Monoclonal Antibodies

In a pilot experiment three monoclonal antibodies (Moabs) against D-antigen of poliovirus were lyophilized using a preservation mixture containing HES, Na-glutamate and trehalose.

Moabs diluted in 0,1 mmol/L PBS pH 7,2 and 1% BSA (w/v) were subsequently 1:1 diluted with the preservation mixture resulting in a Moab formulation containing 10% HES+5% Na-glutamate+20% trehalose. Three moabs, respectively directed against D-antigen of typel poliovirus, type 2 poliovirus or type 3 poliovirus, were used.

The moabs were used to determine the potency (D-antigen Units, DU) of different batches trivalent polio vaccine. Potency titers determined using lyophilized moabs (stored at 20° C. for 3 weeks) were compared with reference moabs stored at −30° C.

TABLE 1
Potency of vaccine batches A, B, C and D determined by lyophilized
moabs (type 1, 2 and 3) relative to respective potencies
determined by reference moabs type 1, 2 and 3)

| Vaccine | Type 1 | Type 2 | Type 3 |
|---|---|---|---|
| A | 99% | 104% | 94% |
| B | 99% | 104% | 100% |
| C | 94% | 103% | 98% |
| D | 96% | 96% | 103% |

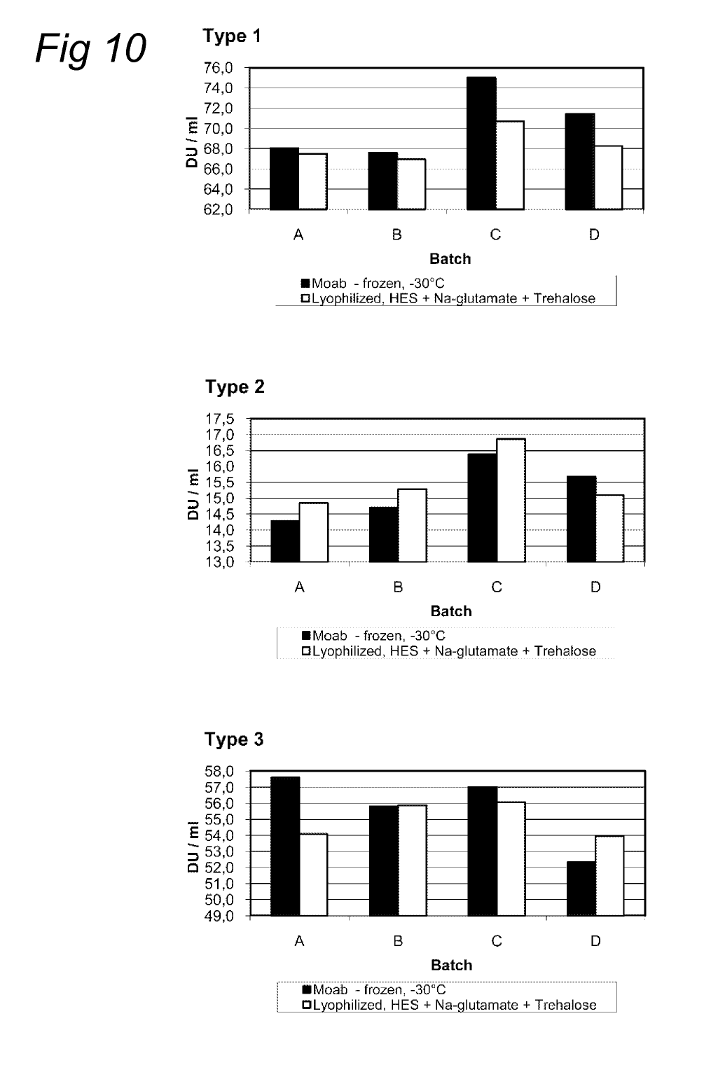

As shown in FIG. 10 and table 1, moab formulations containing 10% HES+5% Na-glutamate+20% trehalose are resistant to lyophilization and when lyophilized can be stored at 20° C. for at least 3 weeks without loss of D-antigen binding.

### Example 5

### Lyophilization of Inactivated Polio Vaccine

In another pilot experiment Trivalent Inactivated Polio Vaccine (IPV; 5 μg/ml; Type1 411DU/ml; Type2 89 DU/ml; Type2 314 DU/ml) was lyophilized. IPV was 1:1 diluted resulting in IPV formulations containing:
10% HES+5% Na-glutamate+20% trehalose
20% trehalose
5% Na-glutamate
10% Na-glutamate
H₂O

After lyophilization, the lyophilized formulations were stored at 4° C. until analyses. The potency (D-antigen Units, DU) of the lyophilized vaccines was determined by a D-antigen ELISA and compared with a reference preparation.

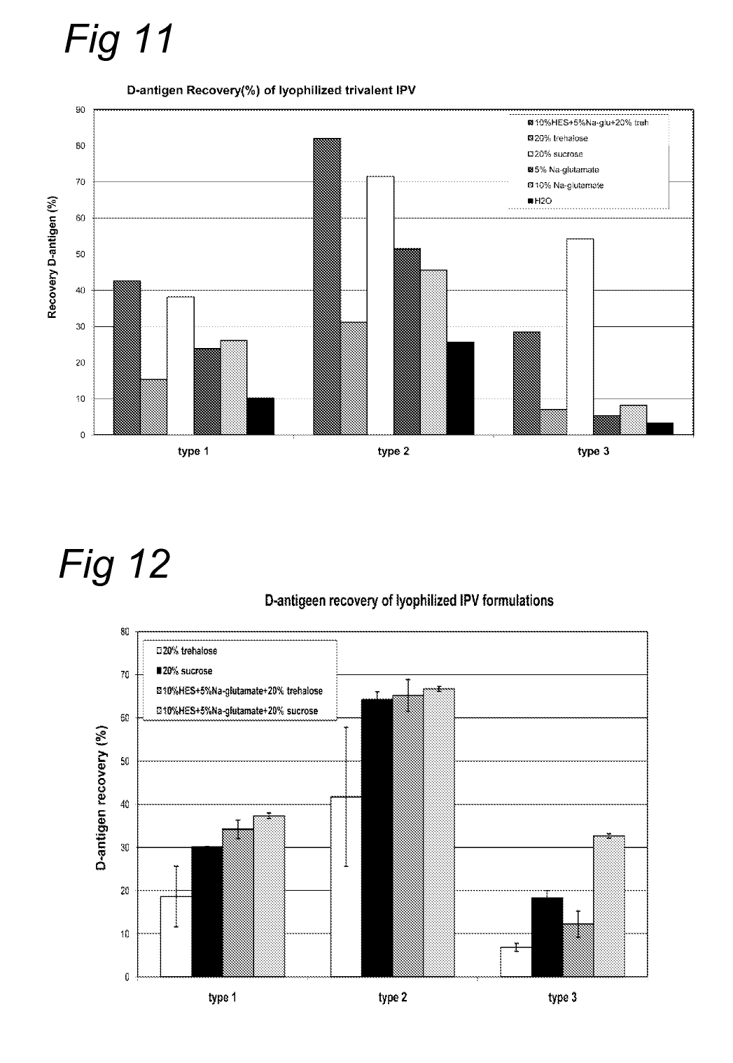

As shown in FIG. 11 IPV is very sensitive to lyophilization stresses. Without the addition of stabilizers 70-95% of the IPV potency is lost. Trehalose and Na-glutamate could increase the recovery to some extent. However, best recovery was found with the IPV formulation tested that contained 10% HES, 5% Na-glutamate and 20% trehalose.

In another pilot experiment lyophilization of another Trivalent Inactivated Polio Vaccine (IPV; 5μg/ml) was evaluated using formulations containing:
20% trehalose
20% Sucrose
10% HES+5% Na-glutamate+20% trehalose
10% HES+5% Na-glutamate+20% sucrose

As shown in FIG. 12, also this IPV-lot is very sensitive to lyophilization stresses. Both trehalose and sucrose could increase the recovery to some extent. However, best recoveries were found for the IPV formulation tested that contained 10% HES, 5% Na-glutamate and 20% sucrose. Especially for IPV type 3.

1. A preservation mixture comprising glutamate, a saccharide and hydroxy-ethyl starch (HES), wherein the mixture does not comprise a non-reducing derivative of a monosaccharide.

2. The mixture according to claim 1, wherein glutamate is sodium glutamate.

3. The mixture according to claim 2, wherein the saccharide is a mono- or disaccharide.

4. A composition comprising the mixture according to claim 1 and a biological component.

5. The composition according to claim 4, wherein the biological component comprises a cell, a micro-organism, a virus, a protein or a derivative thereof.

6. The composition according to claim 5, wherein
(a) the glutamate is present in a concentration between about 0.05% and about 60% w/v,
(b) the saccharide is present in a concentration between about 0.05% and about 60% w/v, and
(c) the composition further comprises a polymer the concentration of which is between about 0.005% and about 50% w/v.

7. The composition according to claim 6, wherein
(a) the glutamate concentration is between about 5% and about 10% w/v,
(b) the saccharide concentration is between about 10% and about 20% w.v, and
(c) the polymer concentration is about 10% w/v.

8. A method of preserving a biological component, comprising:
(a) adding each of the following constituents to, or mixing them with, a biological component:
(i) a glutamate,
(ii) a saccharide, with the proviso that the saccharide is not a non-reducing derivative of a monosaccharide, and
(iii) HES, thereby obtaining a composition in accordance with claim 4, and
(b) drying the composition obtained in (a).

9. The method according to claim 8, wherein the drying is freeze-drying.

10. The mixture according to claim 3, wherein the disaccharide is sucrose or trehalose.

11. The method according to claim 8, wherein the biological component comprises a cell, a micro-organism, a virus, a protein or a derivative thereof.

12. The method according to claim 8 wherein glutamate is sodium glutamate.

13. The method according to claim 12, wherein the saccharide is a mono- or disaccharide.

14. The method according to claim 13, wherein the disaccharide is sucrose or trehalose.

15. The method according to claim 8, wherein
(a) the glutamate is present in a concentration between about 0.05% and about 60% w/v,
(b) the saccharide is present in a concentration between about 0.05% and about 60% w/v, and
(c) the composition further comprises a polymer the concentration of which is between about 0.005% and about 50% w/v.

16. The method according to claim 15, wherein:
(a) the glutamate concentration is between about 5% and about 10% w/v,
(b) the saccharide concentration is between about 10% and about 20% w.v, and
(c) the polymer concentration is about 10% w/v.
